(19) United States
(12) Patent Application Publication
(10) Pub. No.: US 2012/0178094 A1
(43) Pub. Date: Jul. 12, 2012

(54) Method for Categorizing Circulating Tumor Cells

(76) Inventor: Peter Kuhn, Solana Beach, CA (US)

(21) Appl. No.: 13/393,177

(22) PCT Filed: Sep. 2, 2010
(86) PCT No.: PCT/US10/47676

§ 371 Date: Mar. 28, 2012

Related U.S. Application Data
(60) Provisional application No. 61/239,682, filed on Sep. 3, 2009.

(51) Int. Cl. C12Q 1/68; G01N 33/574

(52) U.S. Cl. 435/6.12; 435/7.23

(57) ABSTRACT

The present invention provides method for categorizing circulating tumor cells (CTCs) using various cellular markers and revealing or non-revealing assays which provide beneficial insights for clinical staging and therapy decision making in cancer patients.

### BACKGROUND OF THE INVENTION

1. Field of the Invention

The present invention relates generally to medical diagnostics and more specifically to categorization of circulating tumor cells.

2. Background Information

Circulating tumor cells (CTCs) are generally, although not exclusively, epithelial cells that originate from a solid tumor in very low concentration into the blood stream of patients with various types of cancer. The shedding of CTCs by an existing tumor or metastasis often results in formation of secondary tumors. Secondary tumors typically go undetected and lead to 90% of all cancer deaths. Circulating tumor cells provide the link between the primary and metastatic tumors. This leads to the promise of using the identification and characterization of circulating tumor cells for the early detection and treatment management of metastatic epithelial malignancies. Detection of CTCs in cancer patients offers an effective tool in early diagnosis of primary or secondary cancer growth and determining the prognosis of cancer patients undergoing cancer treatment because number and characterization of CTCs present in the blood of such patients has been correlated with overall prognosis and response to therapy. Accordingly, CTCs serve as an early indicator of tumor expansion or metastasis before the appearance of clinical symptoms.

While the detection of circulating tumor cells (CTCs) has important prognostic and potential therapeutic implications in the management and treatment of cancer, because of their occult nature in the bloodstream, these rare cells are not easily detected. CTCs were first described in the 1800s, however only recent technological advances have allowed their reliable detection. CTCs are thought to exist in peripheral blood at ultra-low concentrations of patients with tumors. For example, for patients with carcinomas it is estimated that every one in ten million normal blood cells is a CTC.

Standard methods for enumeration/characterization of CTCs are immunomagnetic enrichment methods targeting the surface protein EpCam, fiber-optic array scanning technology, and “CTC chip” assays.

Immunomagnetic enrichment technology relies upon immunomagnetic enrichment of tumor cell populations using magnetic ferrofluids linked to an antibody which binds epithelial cell adhesion molecule (EpCAM), expressed only on epithelial derived cells.

In performing Fiber-optic Array Scanning Technology (FAST) to detect CTCs red blood cells are lysed and nucleated cells are distributed as a monolayer on slides that can hold up to 30 million cells. There is no enrichment step in this methodology. Cells are fixed, permeabilized and stained with a pan anti-cytokeratin antibody-Alexa Fluor 555, CD45-Alexa Fluor 647, and DAPI (nuclear stain). FAST scans each slide and identifies the location of each red fluorescent object on the slide. Each fluorescent object is imaged via an automated digital microscope and CTCs are enumerated as being CK+, CD45-, DAPI+ cells.

Another method for enumeration/characterization of CTCs is microfluidic or “CTC-Chip” technology. In this method whole blood flows past 78,000 EpCam-coated microposts. EpCam+ cells stick to the posts and are subsequently stained with cytokeratin, CD45, and DAPI.

Assays for identifying CTCs using epithelial specific markers such as cytokeratin and EpCAM and/or tissue specific markers such as PSA, PSMA, CDX2 and TTF-1 have been described in the art. Assays for revealing and detecting CTCs have also been described, for example as in U.S. Ser. No. 12/553,733, filed Sep. 3, 2009, incorporated herein by reference. Revealing CTCs in a sample includes removing, degrading or altering a protein, carbohydrate, cell, or a combination thereof, aggregated, or in physical association with, the surface of the circulating tumor cells to unmask the cell, thereby revealing the circulating tumor cell. Revealing the cells may include removing, degrading or altering blood plasma proteins, carbohydrates, platelets, other blood cells, or a combination thereof. Typically, the blood plasma protein is a clotting factor, such as fibrin. To unmask the cells, the cells may be treated enzymatically (e.g., biochemical reaction mediated by an enzyme), mechanically (e.g., mechanical force), electrically (e.g., electrical force), electromagnetically (e.g., electromagnetic radiation of the electromagnetic spectrum), chemically, or any combination thereof. The revealed cells may be further analyzed by image analysis and/or detection of cell surface markers.

Advances in methods for revealing, isolating, detecting, identifying and enriching circulating tumor cells (CTCs) are continually being made in the cancer field. However, as improvements are made, improved methods of analysis using the increasing amount of information being gathered regarding CTCs is required. Thus methods for improved clinically meaningful analysis of CTCs for use in clinical, research and development settings as well as innovative methods for the treatment of cancers are necessary.

### SUMMARY OF THE INVENTION

The present invention provides methods for categorizing CTCs using various cellular markers and revealing or non-revealing assays which provide beneficial insights for clinical staging and therapy decision making in cancer patients.

Accordingly, in one aspect, the invention provides a method for categorizing circulating tumor cells (CTCs) from a subject. The method includes: a) providing a first and second sample from a subject; b) revealing CTCs in the first sample; c) analyzing the revealed CTCs of the first sample using a reagent specific for a first cell marker; d) analyzing CTCs of the second sample using a reagent specific for the first or a second cell marker, wherein the CTCs of the second sample are not revealed or unmasked before analysis; and e) comparing the results of c) and d) to provide a categorization of the CTCs from the subject, thereby categorizing the CTCs. The method may further include comparing the results of e) with a prior categorization of CTCs from the subject, or to a known categorization.

The present invention further provides a method for prognosing cancer in a subject. The method includes: a) providing a first and second sample from a subject; b) revealing CTCs in the first sample; c) analyzing the revealed CTCs of the first sample using a reagent specific for a first cell marker; d) analyzing CTCs of the second sample using a reagent specific for the first or a second cell marker, wherein the CTCs of the second sample are not revealed or unmasked before analysis; and e) comparing the results of c) and d) to provide a categorization of the CTCs from the subject; and f) determining a prognosis, thereby prognosing cancer in a subject. The method may further include comparing the results of e) with a prior categorization of CTCs from the subject, or to a known categorization.

The present invention further provides a method of determining responsiveness of a subject to a particular therapeutic regime. The method includes: a) providing a first and second sample from a subject; b) revealing CTCs in the first sample; c) analyzing the revealed CTCs of the first sample using a reagent specific for a first cell marker; d) analyzing CTCs of the second sample using a reagent specific for the first or a second cell marker, wherein the CTCs of the second sample are not revealed or unmasked before analysis; and e) comparing the results of c) and d) to provide a categorization of the CTCs from the subject; and f) determining the responsiveness of the subject to a therapeutic regime. The method may further include comparing the results of e) with a prior categorization of CTCs from the subject, or to a known categorization.

The present invention further provides a method of determining the effectiveness of a candidate agent in the treatment of cancer. The method includes: a) providing a first and second sample from a subject; b) revealing CTCs in the first sample; c) analyzing the revealed CTCs of the first sample using a reagent specific for a first cell marker; d) analyzing CTCs of the second sample using a reagent specific for the first or a second cell marker, wherein the CTCs of the second sample are not revealed or unmasked before analysis; and e) comparing the results of c) and d) to provide a categorization of the CTCs from the subject; and f) determining the effectiveness of the candidate agent in the treatment of cancer. The method may further include comparing the results of e) with a prior categorization of CTCs from the subject, or to a known categorization.

### DETAILED DESCRIPTION OF THE INVENTION

The present invention provides methods for categorizing CTCs using various cellular markers and revealing or non-revealing assays as well as methods of treating and diagnosing cancer.

The revealing methodology unmasks CTCs that have their cellular membrane obstructed by molecules such as fibrin or carbohydrates or by cells such as platelets and white blood cells. It was discovered that a significant number of CTCs in circulation remain undetectable because they are “masked” or “cloaked” by cells, proteins, biomolecules and other factors aggregated at the surface of the CTCs shielding them from surface interactions and/or intracellular antibody binding as an effective immune escape mechanism. For example, platelets, fibrin, and other clotting proteins act as a “cloak device” to mask or veil critical cell surface markers on the surface of the cells allowing them to escape detection or observation using current methods which explain why such few CTCs are detected using current methods. Similarly, other factors can effectuate masking or veiling of CTCs such as, for example, glycosylation of surface protein markers, or attraction of sugar molecules (from entities other than the CTC itself) or association of cell surface components with other biomolecules.

The revealing methodology removes these obstructing molecules and cells, thereby making the CTC specific markers more accessible to marker-specific binding reagents, which are pre-labeled or can be specifically detected with secondary labels or facilitate detection by other means. These detectable reagents are then used to identify the CTC. By performing assays by either applying the revealing step or omitting the revealing step with different combinations of marker-specific binding reagents, it is possible to create new useful information to characterize a tumor and its staging, and to predict prognosis and response to therapy.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

In general, reference to “a circulating tumor cell” is intended to refer to a single cell, while reference to “circulating tumor cells” is intended to refer to more than one cell. However, one of skill in the art would understand that reference to “circulating tumor cells” is intended to include a population of circulating tumor cells including one or more circulating tumor cells.

As discussed further herein, signal intensity of detectable reagents may be utilized to obtain varying types of information useful in categorizing CTCs. For example, from signal intensity, one may deduce the expression level of the protein of interest (e.g., cell specific markers, both intracellular and cell surface markers). Additionally, from signal intensity, one may deduce the level of masking versus unmasking present in a particular sample. Signal intensity of cell surface markers, as well as intracellular markers provides this such information. This is the case for intracellular markers, due to the possibility that the coat (e.g., mask) may be more robust than the cell membrane (e.g., protein mesh is more robust than lipids) and such harsh conditions are required to uncoat the CTC to reveal the CTC that the intracellular environment is degraded or destroyed.

Utilizing signal intensity of various cell markers from CTCs detected in revealing assays and non-revealing assays in specific comparisons provides new useful information which allows categorization of the CTCs to gain insight of a patients cancer allowing prognosis, and therapy decisions.

Accordingly, in one aspect, the invention provides a method for categorizing circulating tumor cells (CTCs) from a subject. The method includes a) providing a first and second sample from a subject; b) revealing CTCs in the first sample; c) analyzing the revealed CTCs of the first sample using a reagent specific for a first cell marker; d) analyzing CTCs of the second sample using a reagent specific for the first or a second cell marker, wherein the CTCs of the second sample are not revealed or unmasked before analysis; and e) comparing the results of c) and d) to provide a categorization of the CTCs from the subject, thereby categorizing the CTCs. The method may further include comparing the results of e) with a prior categorization of CTCs from the subject, or to a known categorization.

The methods of the present invention utilize revealing assays to generate revealed CTCs. As used herein, the terms “revealing” and “revealing for generally pertain to altering a CTC in its natural state so as to make the CTC more amendable to detection, analysis, characterization, and/or further processing, such as enriching. Revealing a CTC may include removing and/or degrading, all or some biomolecules aggregated and/or associated with the surface and/or surface components of the CTC. For example, revealing a CTC may include unmasking or unveiling the CTC by removing, degrading, or altering aggregated cells (e.g., platelets), carbohydrates, and/or proteins (e.g., fibrin) aggregated and/or physically associated with the surface of the CTC allowing access to one or more CTC cellular components, such as surface components, including for example, cancer surface markers and other surface bound cellular components, as well as intracellular components, such as nucleic acids and other intracellular components (e.g., nuclear and cytosolic proteins, and the like). As such, “unmasking” and/or “unveiling” are intended to include altering a feature of a CTC in its natural state that may assist in cloaking the CTC from immune recognition or response by the host and/or making the CTC more amendable to detection, analysis, characterization, and/or further processing. Revealing a CTC may include altering a CTC cellular component, such as an epitope of a cell surface marker, or protein physically associated and/or aggregated with the CTC.

Additionally, the methods of the present invention utilize detection of CTCs from non-revealing assays. As used herein, CTCs from non-revealing assays is intended to mean that the CTCs are not unmasked or revealed as is the case for CTCs from a revealing assay. For example, a CTC from a non-revealing assay is not unmasked before detection of a cell marker and therefore the cell marker is detected without enzymatic or other treatment to unmask the CTC; but rather is detected in its generally natural state.

The term “biomolecule” is intended to generally refer to any organic or biochemical molecule that occurs in a biological system.

CTCs may be revealed in any suitable sample type. As used herein, the term “sample” refers to any sample suitable for the methods provided by the present invention. The sample may be any sample that includes CTCs suitable for detection. Sources of samples include whole blood, bone marrow, pleural fluid, peritoneal fluid, central spinal fluid, urine, saliva and bronchial washes. In one aspect, the sample is a blood sample, including, for example, whole blood or any fraction or component thereof A blood sample, suitable for use with the present invention may be extracted from any source known that includes blood cells or components thereof, such as veinous, arterial, peripheral, tissue, cord, and the like. For example, a sample may be obtained and processed using well known and routine clinical methods (e.g., procedures for drawing and processing whole blood). In one aspect, an exemplary sample may be peripheral blood drawn from a subject with cancer.

The term “blood component” is intended to include any component of whole blood, including red blood cells, white blood cells, platelets, endothelial cells, mesotheial cells or epithelial cells. Blood components also include the components of plasma, such as proteins, lipids, nucleic acids, and carbohydrates, and any other cells that may be present in blood, due to pregnancy, organ transplant, infection, injury, or disease.

The term “cancer” as used herein, includes a variety of cancer types which are well known in the art, including but not limited to, dysplasias, hyperplasias, solid tumors and hematopoietic cancers. Many types of cancers are known to metastasize and shed circulating tumor cells or be metastatic, for example, a secondary cancer resulting from a primary cancer that has metastasized. Additional cancers may include, but are not limited to, the following organs or systems: brain, cardiac, lung, gastrointestinal, genitourinary tract, liver, bone, nervous system, gynecological, hematologic, skin, breast, and adrenal glands. Additional types of cancer cells include gliomas (Schwannoma, glioblastoma, astrocytoma), neuroblastoma, pheochromocytoma, paraganlioma, meningioma, adrenalcortical carcinoma, medulloblastoma, rhabdomyoscarcoma, kidney cancer, vascular cancer of various types, osteoblastic osteocarcinoma, prostate cancer, ovarian cancer, uterine leiomyomas, salivary gland cancer, choroid plexus carcinoma, mammary cancer, pancreatic cancer, colon cancer, and megakaryoblastic leukemia; and skin cancers including malignant melanoma, basal cell carcinoma, squamous cell carcinoma, Karposi's sarcoma, moles dysplastic nevi, lipoma, angioma, dermatofibroma, keloids, sarcomas such as fibrosarcoma or hemangiosarcoma, and melanoma.

The term “circulating tumor cell” (CTC) is intended to mean any cancer cell that is found in a subject's sample. Typically CTCs have been exfoliated from a solid tumor. As such, CTCs are often epithelial cells shed from solid tumors found in very low concentrations in the circulation of patients with advanced cancers. CTCs may also be mesothelial from sarcomas or melanocytes from melanomas.

As used herein, a cellular component is intended to include any component of a cell that may be at least partially isolated upon lysis of the cell. Cellular components may be organelles, such as nuclei, perinuclear compartments, nuclear membranes, mitochondria, chloroplasts, or cell membranes; polymers or molecular complexes, such as lipids, polysaccharides, proteins (membrane, trans-membrane, or cytosolic); nucleic acids, viral particles, or ribosomes; or other molecules, such as hormones, ions, cofactors, or drugs.

Revealed CTCs are unmasked and/or altered from their natural state. As discussed herein, the CTCs may be unmasked and revealed by removing, degrading, and/or altering aggregated cells (e.g., platelets), carbohydrates, and/or proteins (e.g., fibrin) allowing access to critical components of the CTC critical to detection and/or analysis, such as, but not limited to surface components such as cancer markers and other surface bound cellular components.

Accordingly, a sample including revealed CTCs or a revealed CTC population is intended to mean a sample in which the sample has been processed as described herein to increase the relative population of revealed (e.g., unmasked and/or altered) CTCs as compared to if the sample had not been processed, for example, relative to a non-revealed sample. For example, the relative population of revealed CTCs in a sample may be increased by at least about 10%, 25%, 50%, 75%, 100% or by a factor of at least 2, 5, 10, 20, 30, 40, 50, 60, 70, 80, 90, 100, or even 200.

Revealing assays to unmask CTCs include removing, degrading or altering a protein, carbohydrate, cell, or a combination thereof, aggregated, or in physical association with, the surface of the circulating tumor cells to unmask the cell, thereby revealing the circulating tumor cells. Revealing the cells include removing, degrading or altering blood plasma proteins, carbohydrates, platelets, other blood cells, or a combination thereof. For example, blood plasma factor that are clotting factors, such as fibrin may be removed to reveal and unmask CTCs.

CTCs may be revealed and unmasked using a variety of methods. For example, CTCs may be revealed using methods including treatments such as, but not limited to, enzymatic, mechanical, electrical, electromagnetic radiation, or chemical treatment, or any combination thereof.

In one embodiment, removal and/or degradation of the proteins and/or cells from the surface of a CTC is performed by treating the CTCs enzymatically. Enzymatic treatment may occur by fibrinolysis. In a related method, fibrinolysis is produced by enzymatic activation of plasminogen.

As used herein, fibrinolysis is intended to mean the enzymatic process wherein fibrin and/or products of coagulation, such as fibrin clots and the like are degraded. In one aspect, degradation by fibrinolysis is performed by treatment of CTCs with the enzyme plasmin. Plasmin is a serine protease present in the blood that degrades fibrin as well as other blood plasma proteins performing a crucial role in fibrinolysis. Plasmin is known to enzymatically cleave such proteins as fibrin, fibronectin, thrombospondin, laminin, and von Willebrand factor. A variety of natural and synthetic plasmins are well known in the art and may be used with the methods of the present invention so long as the enzyme retains some role in fibrinolysis.

Plasmin is derived from plasminogen which is excreted from the liver into the circulation. Once in the circulation, plasminogen may be activated by a variety of factors to generate plasmin, such as tissue plasminogen activator (tPA), urokinase plasminogen activator (uPA), thrombin, fibrin, and factor XII (Hageman factor). Accordingly, in another aspect of the invention, fibrinolysis is produced by enzymatic activation of plasminogen.

Fibrinolysis may also be effectuated by other naturally or synthetically occurring agents. For example, in yet another aspect of the invention fibrinolysis may occur by treatment of CTCs with a natural or synthetic animal venom or toxin. For example, venomous animals, such as but not limited to bats, snakes and insects are known to possess venom or toxins capable of direct or indirect enzymatic activation of fibrinolysis.

In addition to enzymatic degradation cells and proteins aggregated to the surface of masked CTCs, CTCs may be treated mechanically, electrically, or chemically. For example, mechanical forces may be used in the treatment of CTCs to shear cells and proteins aggregated to the surface. Accordingly, the present invention envisions treating CTCs with any type of mechanical force or movement capable of unmasking CTCs. Additionally, treatment with a variety of electrical forces may be utilized to unmask CTCs such as, but not limited to, electromagnetic, electrostatic, electrochemical, electromagnetic radiation, ultrasonic forces, and the like. Electromagnetic radiation may include application of radiation from any region of the electromagnetic spectrum.

Further, treatment with a variety of chemical agents may be utilized to reveal CTCs. For example, chemical agents such as, but not limited to, natural or synthetic molecules, organic compounds, non-organic compounds, drugs, therapeutics, and the like may be used to activate or inhibit various steps in the fibrinolysis pathway leading to degradation of clotting factors. Additional chemical agents that may be used to unmask CTCs include anti-platelets, anti-coagulants and/or blood thinners which degrade and/or suppress the platelet and fibrin activation on the surface of CTCs. Common anti-platelets, anti-coagulants and blood thinners that may be used include but are not limited to, cyclooxygenase inhibitors, such as aspirin; adenosine diphosphate (ADP) receptor inhibitors, such as clopidogrel, and ticlopidine; phosphodiesterase inhibitors, such as cilostazol; glycoprotein IIB/IIIA inhibitors, such as abciximab, eptifibatide, tirofiban, and defibrotide; adenosine reuptake inhibitors such as dipyridamole; vitamin K antagonists; heparin and heparin derivative substances; clopidogrel (Plavix™); benzopyrone (coumarin); and direct thrombin inhibitors. In an exemplary aspect, the CTCs are treated with heparin to reveal for the cells.

In one embodiment, mechanical forces sufficient to reveal CTCs by breaking up agglomerated cells in physical association with the surface of CTCs may be generated in microfluidic devices used for biomedical and diagnostic research. The microscale devices that constitute a microfluidic system typically consist of a plurality of posts, grooves or microchannels, and chambers etched or molded in a substrate commonly composed of silicon, plastic, quartz, glass, or plastic. The size, shape, configuration of these microscale features, as well as their interconnections determine the physical forces generated on the constituents of a fluid sample flowing through the device, such as cells or clusters of cells suspended in the fluid. It is envisioned that the microscale features of a microfluidic device, along with factors, such as rate of fluid flow, may be configured and exploited to generate sufficient mechanical forces to reveal CTCs in a fluid sample. Additionally, one of skill in the art would recognize that CTCs may be treated with one or more other treatment techniques (e.g., enzymatically, chemically, electrically, and like), separate from, or in addition to the mechanical forces in the microfluidic system. Accordingly, CTCs may be treated enzymatically, chemically, or the like, before or after being introduced into a microfluidic device, as well as in the microfluidic device itself.

In various aspects, it may be necessary to limit the duration of treatment to prevent excess degradation that may impair the integrity of the CTCs leading to lysis. Accordingly, in various embodiments, cells should be treated for a time sufficient for removing molecules from the CTCs so that the cell can be further detected and/or identified. While this time may vary depending of the type of treatment applied to the cell, it is within the knowledge of one skilled in the art to determine such time by routine assay. Additionally, where CTCs are treated enzymatically or chemically, reactions may be controlled by addition of specific inhibitors to slow or stop reactions.

The total number of revealed CTCs included in a revealed CTC population is dependent, in part, on the initial sample volume. In various aspects, revealing of CTCs in a wide range of initial sample volumes is sufficient to produce a revealed number of CTCs capable of providing clinically significant results. As such, the initial sample volume may be less than about 25 μl, 50 μl, 75 μl, 100 μl, 125 μl, 150 μl, 175 μl, 200 μl, 225 μl, 250 μl, 300 μl, 400 μl, 500 μl, 750 μl, 1 ml, 2 ml, 3 ml, 4 ml, 5 ml, 6 ml, 7 ml, 8 ml, 9 ml or greater than about 10 ml. In an exemplary aspect, the initial sample volume is between about 100 and 200 μl. In another exemplary aspect, a sample processed as described herein includes greater than about 1, 2, 5, 7, 10, 15, 20, 30, 40, 50, 100, 200, 300, 400, 500, 600, 700, 800, 900, or even 1000 revealed CTCs.

In various embodiments of the present invention, revealed and unrevealed CTCs are analyzed to derive clinically significant data. Analysis of CTCs may be performed by a variety of methods depending of the type of data desired. For example, in various aspects, CTCs may be analyzed by detecting and characterizing the CTCs via assays utilizing recognition and/or binding of cellular components, such as cell surface markers. A variety of detection/immobilization assays are contemplated for use with the present invention from which useful data may be derived. Additional analysis methods may include image analysis.

As used herein, image analysis includes any method which allows direct or indirect visualization of CTCs. For example, image analysis may include, but not limited to, ex vivo microscopic or cytometric detection and visualization of cells bound to a solid substrate, flow cytometry, fluorescent imaging, and the like. In an exemplary aspect, CTCs are detected using antibodies directed to cell surface markers and subsequently bound to a solid substrate and visualized using microscopic or cytometric detection.

In various embodiments, a variety of cell markers may be used to analyze, detect and categorize CTCs. As used herein, cell markers include any cellular component that may be detected within or on the surface of a cell, or a macromolecule bound or aggregated to the surface of the cell. As such, cell markers are not limited to markers physically on the surface of a cell. For example, cell markers may include, but are not limited to surface antigens, transmembrane receptors or coreceptors, macromolecules bound to the surface, such as bound or aggregated proteins or carbohydrates, internal cellular components, and the like. In one aspect, the cell markers may be a cell adhesion molecule, such as EpCAM or a cytokeratin. In an exemplary aspect, the antibodies used to detect cell markers are anti-cytokeratin, pan-keratin and anti-EpCAM.

Additionally, a number of cell markers known to be specific to cancers may be targeted or otherwise utilized to detect and analyze CTCs. For example, various receptors have been found to be expressed or over expressed only in particular type of cancers. In various aspects of the invention cell markers include EGFR, HER2, ERCC1, CXCR4, EpCAM, E-Cadherin, Mucin-1, Cytokeratin, PSA, PSMA, RRM1, Androgen Receptor, Estrogen Receptor, Progesterone Receptor, IGF1, cMET, EML4, or Leukocyte Associated Receptor (LAR). Further, cell markers may be utilized that are specific to particular cell types. For example, useful endothelial cell surface markers include CD105, CD106, CD144, and CD146, while useful tumor endothelial cell surface markers include TEM1, TEM5, and TEM8.

In various embodiments, the methods of the present invention may include further processing of the CTCs of the patient samples prior to analysis. For example, in one embodiment, the CTCs are captured by techniques commonly used to enrich a sample for CTCs, for example those involving immunospecific interactions, such as immunomagnetic capture. A variety of immunocapture methods are known, including immunocapture with beads or posts. A magnetic field or solid supports may aid the immunocapture. Various cell markers may be used for immunocapture, including EGFR, HER2, ERCC1, CXCR4, EpCAM, E-Cadherin, Mucin-1, Cytokeratin, PSA, PSMA, RRM1, Androgen Receptor, Estrogen Receptor, Progesterone Receptor, IGF1, cMET, EML4, or Leukocyte Associated Receptor (LAR).

Immunomagnetic capture, also known as immunomagnetic cell separation typically involves attaching antibodies directed to proteins found on a particular cell type to small paramagnetic beads. When the antibody-coated beads are mixed with a sample, such as blood, they attach to and surround the particular cell. The sample is then placed in a strong magnetic field, causing the beads to pellet to one side. After removing the blood, captured cells are retained with the beads. Many variations of this general method are well known in the art and suitable for use to enrich the CTCs after they have been revealed using methods of the present invention.

In another embodiment, the CTCs are further processed prior to an enrichment step using filtration. The process of revealing the CTCs breaks down aggregates of cells, thereby making the filtration more efficient.

In another embodiment, the CTCs are further processed via cell separation by density gradient sedimentation. Typically, the process relies on a gross physical distinction, such as cellular density for separating nucleated cells such as CTCs from erythrocytes and other non-CTC cells. Many variations of this general method are well known in the art and suitable for use to enrich the CTCs after they have been revealed using methods of the present invention.

In another embodiment, the CTCs are enriched by a technique called “panning”. Typically, such processes utilize an antibody specific to the cell type in question in which the antibody is adhered to a solid surface. The cell mixture is layered on top of the antibody-coated surface, the targeted cells tightly adhere to the solid surface due to the immunospecific interaction involving antibody-antigen binding. Non-adherent cells are rinsed off the surface, thereby effecting a cell separation and enrichment. Cells that express a cell surface protein recognized by the antibody are retained on the solid surface whereas other cell types are not.

Detection and analysis of cell markers may be performed in a variety of ways. In an exemplary embodiment, detection and analysis is performed using fluorescent microscopy and image analysis employing slides.

Typically, one would perform the different combinations of marker-specific binding reagents in separate reactions. Those reactions would occur on individual slides. However, one could also perform the different combinations of marker-specific binding reagents on the same slide by using labels that are distinct between the different reagents, for instance different fluorescent dyes that excite and emit in different wavelengths.

After labeling, slides may be imaged and with computers and information processing algorithms, the CTCs are identified and counted. These images may then be reviewed by trained technicians or pathologists to perform analysis of the specific cell markers detected on the sample processed using the reveal assay and the sample not processed using the reveal assay.

Analysis utilizes mathematical operations on the various CTC numbers to categorize, characterize and predict prognosis and response. In one embodiment, ratios provide useful information.

While one of skill in the are would appreciate that any number of cell markers may be utilized as discussed herein, the following table exemplifies use of the cell markers Cytokeratin and EpCAM. For instance, using the data in Table 1, the percentage of Cytokeratin (+) cells that are detected in the Reveal (+) assay compared to the Reveal (−) assay is B/A. Similarly, the percent increase in Cytokeratin (+) cells from the Reveal (+) assay is (B−A)/A. These same operations may be applied to the EpCAM data where D/C is the percentage of EpCAM (+) cells in Reveal (+)/Reveal (−) and (D−C)/C is the percent increase. The ratio of EpCAM (+) cells to Cytokeratin (+) cells under each Reveal condition is also useful in characterizing and predicting tumor response. In this case, the ratios would be C/A and D/B. These particular mathematical operations are only exemplary as many different mathematical operations are possible when analyzing the data.

TABLE 1
Demonstrative use of the combination
of assays to create new information.

| | Reveal (−) | Reveal (+) |
|---|---|---|
| Cytokeratin (+) | A | B |
| EpCAM (+) | C | D |

A, B, C and D are the numbers of CTCs enumerated under each condition.

Categorization of CTCs, using the methods of the invention, is useful in assessing cancer prognosis and in monitoring therapeutic efficacy for early detection of treatment failure that may lead to disease relapse. In addition, CTC analysis according to the invention enables the detection of early relapse in presymptomatic patients who have completed a course of therapy. This is possible because the presence of CTCs has been associated and/or correlated with tumor progression and spread, poor response to therapy, relapse of disease, and/or decreased survival over a period of time. Thus, enumeration and characterization of revealed CTCs provides methods to stratify patients for baseline characteristics that predict initial risk and subsequent risk based upon response to therapy.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

Accordingly, in another embodiment, the invention provides a method for prognosing cancer in a subject. The method includes categorizing CTCs as described herein to prognose cancer in the subject. As such, the methods of the present invention may be used, for example, to evaluate cancer.

EpCAM expression in the primary tumor has previously been associated with prognosis. Specifically, high levels of EpCAM expression indicate a worse prognosis. However, primary biopsies are often not available to assess EpCAM expression and/or time may have transpired since a primary biopsy during which the patient may have received treatment and the tumor may have changed over the time course and/or in response to the treatment. CTC analysis offers a minimally invasive way to assess EpCAM expression during the course of disease. By performing the assay matrix above, one assesses the relative EpCAM expression by looking at the ratio of EpCAM:Cytokeratin (C/A and D/B). It is thought that a higher ratio is a negative prognostic indicator and predicts poorer response to therapy.

Masking with proteins, carbohydrates and cells is a survival mechanism for CTCs in the circulatory system. These masking techniques help the CTCs evade the immune system, allowing them time to find a suitable environment to lodge, extravasate, and form a metastatic lesion. As a diagnostic aid, determining the level of masking provides information on prognosis and predicts response to therapy. It is expected that any ratios determined may provide useful insight and assist in determining a prognosis. For example, using the table above, the relative masking numbers for Cytokeratin (B/A) and for EpCAM (D/C) provides this information, where a higher ratio is a negative prognostic indicator and predicts poorer response to therapy.

Exemplary marker-specific binding reagents include cell markers such as EGFR, HER2, ERCC1, CXCR4, EpCAM, E-Cadherin, Mucin-1, Cytokeratin, PSA, PSMA, RRM1, Androgen Receptor, Estrogen Receptor, Progesterone Receptor, IGF1, cMET, EML4, or Leukocyte Associated Receptor (LAR).

The above approach may be generalized. By performing multiple different CTC assays on the same patient, one creates results that provide unique data. When that data is compared to each other, one may create new information that details tumor characterization and predicts patient prognosis and treatment efficacy. Importantly, the information generated is not required to be specific to the marker that is used to establish the comparison ratio, for example, it may be the ratio in itself that is significant.

As such, in various aspects, analysis of a subject's CTC number and categorization may be made over a particular time course in various intervals to assess a subject's progression and pathology. For example, analysis may be performed at regular intervals such as one day, two days, three days, one week, two weeks, one month, two months, three months, six months, or one year, in order to track level and characterization of circulating epithelial cells as a function of time.

It is expected that the measured ratios will likely change over time, for example, in response to treatment or as a result of pathogenesis. The change over time would clearly have different and/or additional interpretational value than assays take on the same patient at a single time point. Accordingly, in one embodiment, the invention includes comparing assays performed at multiple (e.g., 2, 3, 4, 5, 6, 7, 8, 9, 10 or more) time points.

In the case of existing cancer patients, this provides a useful indication of the progression of the disease and assists medical practitioners in making appropriate therapeutic choices based on the increase, decrease, or lack of change in circulating epithelial cells, such as the presence of CTCs in the patient's bloodstream. Any increase, be it 2-fold, 5-fold, 10-fold or higher, in the revealed CTCs over time decreases the patient's prognosis and is an early indicator that the patient should change therapy. Similarly, any increase, be it 2-fold, 5-fold, 10-fold or higher, indicates that a patient should undergo further testing such as imaging to further assess prognosis and response to therapy. Any decrease, be it 2-fold, 5-fold, 10-fold or higher, in the revealed CTCs over time shows disease stabilization and a patient's response to therapy, and is an indicator to not change therapy. For those at risk of cancer, a sudden increase in the number of circulating epithelial cells detected may provide an early warning that the patient has developed a tumor thus providing an early diagnosis. In one embodiment, the detection of revealed CTCs increases the staging of the cancer.

Categorization of CTCs as described herein provides data sufficient to make determinations of responsiveness of a subject to a particular therapeutic regime, or for determining the effectiveness of a candidate agent in the treatment of cancer. Accordingly, the present invention provides a method of determining responsiveness of a subject to a particular therapeutic regime or determining the effectiveness of a candidate agent in the treatment of cancer by categorizing CTCs as described herein. For example, once a drug treatment is administered to a patient, it is possible to determine the efficacy of the drug treatment using the methods of the invention. For example, a sample taken from the patient before the drug treatment, as well as one or more cellular samples taken from the patient concurrently with or subsequent to the drug treatment, may be processed using the methods of the invention. By comparing the results of the analysis of each processed sample, one may determine the efficacy of the drug treatment or the responsiveness of the patient to the agent. In this manner, early identification may be made of failed compounds or early validation may be made of promising compounds.

Four important indicators that provide insight to the clinical activity of candidate compounds include HER2, EGFR, CXCR4, and EphB4 RTK. HER2 provides an indicator of malignancy of a cell by determining mRNA stability and subcellular localization of HER2 transcripts. The resistance of EGFR to acquire mutations, and/or the mutations acquired provides important indicators of the activity of a candidate compound in addition to possible alternative compounds that may be used in combination with the candidate compound. An assessment of the level of DNA repair interference induced with platinum provides insight as to the status of the CXCR4 marker and metastatic condition. Additionally, assessment of the status of Ephβ34 receptor tyrosine kinase provides insight as to the metastatic potential of the cell. Accordingly, using the methods of the present invention, patients taking such candidate drugs may be monitored by taking frequent samples of blood and determining the number of circulating epithelial cells, for example CTCs, in each sample as a function of time. A further analysis of the Her2, EGFR, CXCR4, and Ephβ4 RTK indicators provides information as to pathology of the cancer and efficacy of the candidate drug. Similarly, ERRC1, Cytokeratin, PSA, PSMA, RRM1, Androgen Receptor, Estrogen Receptor, Progesterone Receptor, IGF1, cMET, EML4 and others provide insight into the clinical activity of candidate compounds. The analysis of these indicators of clinical activity may be through immunohistochemistry, fluorescent in situ hybridization (FISH), sequencing, genotyping, gene expression or other molecular analytical technique.

In any of the methods provided herein, additional analysis may also be performed to characterize CTCs, to provide additional clinical assessment. For example, in addition to image analysis and bulk number measurements, PCR techniques may be employed, such as multiplexing with primers specific for particular cancer markers to obtain information such as the type of tumor, from which the CTCs originated, metastatic state, and degree of malignancy. Additionally, cell size, DNA or RNA analysis, proteome analysis, or metabolome analysis may be performed as a means of assessing additional information regarding characterization of the patient's cancer. In various aspects, analysis includes antibodies directed to or PCR multiplexing using primers specific for one or more of the following markers: EGFR, HER2, ERCC1, CXCR4, EpCAM, E-Cadherin, Mucin-1, Cytokeratin, PSA, PSMA, RRM1, Androgen Receptor, Estrogen Receptor, Progesterone Receptor, IGF1, cMET, EML4, or Leukocyte Associated Receptor (LAR).

Although the invention has been described with reference to the above example, it will be understood that modifications and variations are encompassed within the spirit and scope of the invention. Accordingly, the invention is limited only by the following claims.

1. A method for categorizing circulating tumor cells (CTCs) from a subject comprising:
a) providing a first and second sample from a subject;
b) revealing CTCs of the first sample by removing or altering a protein, carbohydrate, cell, or a combination thereof, in physical association with the surface of the CTC, thereby unmasking and revealing the CTC;
c) analyzing the revealed CTCs of b) using a reagent specific for a first cell marker;
d) analyzing CTCs of the second sample using a reagent specific for the first or a second cell marker; and
e) comparing the results of c) and d) to provide a categorization of the CTCs from the subject, thereby categorizing the CTCs.

2. The method of claim 1, further comprising comparing the results of e) with a prior categorization of the subject or a known categorization.

3. The method of claim 1, wherein the CTCs of the second sample are not revealed prior to analysis.

4. The method of claim 1, wherein the comparison of (e) comprises calculation of a ratio of the number of CTCs of the first sample having the first cell marker as compared to the number of CTCs of the second sample having the first cell marker.

5. The method of claim 1, wherein the comparison of (e) comprises calculation of a ratio of the number of CTCs of the first sample having the first cell marker as compared to the number of CTCs of the second sample having the second cell marker.

6. The method of claim 1, wherein the first and second cell marker are selected from the group consisting of: EGFR, HER2, ERCC1, CXCR4, EpCAM, E-Cadherin, Mucin-1, Cytokeratin, PSA, PSMA, RRM1, Androgen Receptor, Estrogen Receptor, Progesterone Receptor, IGF1, cMET, EML4, or Leukocyte Associated Receptor (LAR).

7. The method of claim 1, wherein the reagents of (c) and (d) are antibodies are used to detect the cell markers.

8. The method of claim 7, wherein the antibodies are fluorescently labeled.

9. The method of claim 8, wherein the antibodies are directed to EpCAM, Cytokeratin, or a combination thereof.

10. The method of claim 1, wherein the revealing of (b) comprises removing all or portions of blood plasma proteins, platelets, or a combination thereof.

11. The method of claim 10, wherein the blood plasma protein is a clotting factor.

12. The method of claim 11, wherein the clotting factor is fibrin.

13. The method of claim 1, wherein the revealing of (b) comprises enzymatic treatment, mechanical treatment, electrical treatment, electromagnetic treatment, chemical treatment, or any combination thereof.

14. The method of claim 13, wherein the revealing of (b) comprises enzymatic treatment.

15. The method of claim 14, wherein the enzymatic treatment is by fibrinolysis.

16. The method of claim 15, wherein fibrinolysis is with plasmin.

17. The method of claim 14, wherein enzymatic treatment is by incubation with an animal venom or a toxin.

18. The method of claim 14, wherein enzymatic treatment is by activation of plasminogen.

19. The method of claim 1, wherein the revealing of (b) comprises treatment of the cells with an anti-coagulant or a blood thinner.

20. The method of claim 1, wherein the first and second samples are about 200 microliters.

21. The method of claim 1, further comprising enriching the first or second sample prior to analyzing.

22. The method of claim 21, wherein the samples are enriched immunomagnetically or by filtration.

23. The method of claim 1, wherein the analyzing of (c) or (d) comprises image analysis.

24. The method of claim 23, wherein the image analysis is performed by microscopy or flow cytometry.

25. The method of claim 1, further comprising providing a prognosis to the subject.

26. The method of claim 1, wherein the subject is known to have cancer.

27. The method of claim 26, wherein the subject is undergoing cancer therapy.

28. The method of claim 27, wherein the therapy is chemotherapy.

29. A method for prognosing cancer in a subject comprising:
a) providing a first and second sample from a subject;
b) revealing CTCs of the first sample by removing or altering a protein, carbohydrate, cell, or a combination thereof, in physical association with the surface of the CTC, thereby unmasking and revealing the CTC;
c) analyzing the revealed CTCs of b) using a reagent specific for a first cell marker;
d) analyzing CTCs of the second sample using a reagent specific for the first or a second cell marker;
e) comparing the results of c) and d) to provide a categorization of the CTCs from the subject; and
f) determining a prognosis, thereby prognosing cancer in a subject.

30. The method of claim 29, further comprising comparing the results of e) with a prior categorization of the subject or a known categorization.

31. The method of claim 29, wherein the cells of the second sample are not revealed prior to analysis.

32. A method for determining responsiveness of a subject to a therapeutic regime comprising:
a) providing a first and second sample from a subject;
b) revealing CTCs of the first sample by removing or altering a protein, carbohydrate, cell, or a combination thereof, in physical association with the surface of the CTC, thereby unmasking and revealing the CTC;
c) analyzing the revealed CTCs of b) using a reagent specific for a first cell marker;
d) analyzing CTCs of the second sample using a reagent specific for the first or a second cell marker;
e) comparing the results of c) and d) to provide a categorization of the CTCs from the subject; and
f) determining the responsiveness of the subject to a therapeutic regime.

33. The method of claim 32, further comprising comparing the results of e) with a prior categorization of the subject or a known categorization.

34. The method of claim 32, wherein the cells of the second sample are not revealed prior to analysis.

35. A method for determining effectiveness of a candidate agent in the treatment of cancer comprising:
a) providing a first and second sample from a subject;
b) revealing CTCs of the first sample by removing or altering a protein, carbohydrate, cell, or a combination thereof, in physical association with the surface of the CTC, thereby unmasking and revealing the CTC;
c) analyzing the revealed CTCs of b) using a reagent specific for a first cell marker;
d) analyzing CTCs of the second sample using a reagent specific for the first or a second cell marker;
e) comparing the results of c) and d) to provide a categorization of the CTCs from the subject; and
f) determining the effectiveness of the candidate agent in the treatment of cancer.

36. The method of claim 35, further comprising comparing the results of e) with a prior categorization of the subject or a known categorization.

37. The method of claim 35, wherein the cells of the second sample are not revealed prior to analysis.
